(19) United States
(12) Patent Application Publication
(10) Pub. No.: US 2014/0335526 A1
(43) Pub. Date: Nov. 13, 2014

(54) QUANTIFICATION OF RESIDUAL HOST CELL DNA BY REAL-TIME QUANTITATIVE PCR

(71) Applicants: Alex Garvin, Durmenach (FR); Ralf Holzinger, Rixheim (FR); Nicolas Weber, Rixheim (FR); Anja Fritsch, Weil Am Rhein (DE)

(72) Inventors: Alex Garvin, Durmenach (FR); Ralf Holzinger, Rixheim (FR); Nicolas Weber, Rixheim (FR); Anja Fritsch, Weil Am Rhein (DE)

(73) Assignee: CONFARMA FRANCE, HOMBOURG (FR)

(21) Appl. No.: 13/819,838

(22) PCT Filed: Sep. 5, 2011
(86) PCT No.: PCT/EP2011/065272

§ 371 Date: Feb. 17, 2014

Related U.S. Application Data
(60) Provisional application No. 61/402,704, filed on Sep. 3, 2010.

(51) Int. Cl. C12Q 1/68

(52) U.S. Cl. CPC C12Q 1/6888
USPC 435/6.12

(57) ABSTRACT

The present invention relates to a method for quantifying residual host cell genomic DNA in recombinant protein biologics using quantitative real time PCR.

### FIELD OF THE INVENTION

The present invention relates to the field of analytical molecular biology and specifically to the field of DNA quantitation. In particular, the present invention relates to quantitation of residual host cell DNA in recombinant protein biologics using quantitative real time PCR.

### BACKGROUND OF THE INVENTION

Recombinant protein drugs are inevitably contaminated with genomic DNA from the cells used to make the protein, and both World Health Organization and the Food and Drug Administration (FDA) have recommended that the amount of contaminating DNA does not exceed 10 nanograms of residual DNA per dose of a biologic drug. Furthermore, the FDA recommends that detection methods used for residual DNA be sensitive to at least 10 picograms per dose. Therefore, drug makers are obliged to quantitate the amount of residual DNA in their biologics using highly sensitive methods that can accurately quantitate genomic DNA in the low picogram range.

The most common method now used by the pharmaceutical industry to quantitate residual DNA is the Threshold Method (Molecular Devices Corporation, Menlo Park, Calif., USA). The Threshold Method uses a Sandwich Elisa reaction with a single stranded DNA binding protein and antibodies that recognize single stranded DNA to quantitate DNA from any source in the low picogram range. The non-specificity of this assay is advantageous because it can be used to detect contaminating DNA from any host cell, but the method does have disadvantages in terms of cost, complexity, and reproducibility. Therefore alternative methods to quantitate trace amounts of DNA have been developed.

Quantitative PCR (or qPCR) is another method for measuring trace amounts of DNA. qPCR is a method that measures the fluorescent signal generated in a PCR reaction at each cycle of the reaction. The amount of test sequence introduced into a qPCR reaction will determine the intensity of the signal generated during cycling, and by comparing this signal to that obtained from known standards, DNA can be accurately quantitated over a wide range of concentrations.

The sensitivity of the assay when measuring genomic DNA is directly dependent on the copy number of the target sequence in the genome of the host species, thus highly repetitive sequences are the best targets when high sensitivity is required. The sensitivity is also inversely proportional to the genome size, so that host DNA from organisms with a small genome, such as E. coli (genome size 4.6 megabases), can be quantitated in the low picogram range without resorting to high copy number targets. However, the current trend in bio-pharmaceuticals is to produce protein products in cells from higher eukaryotes such as hamsters, monkeys, and humans which all have genome sizes in excess of 2 gigabases. Therefore, any qPCR assay for residual DNA must be able to detect low picogram amounts of genomic DNA from cells that have a large genome size.

The common approach to obtain the required sensitivity for eukaryotic host cell DNA detection is to use highly repetitive sequence targets. Letwin and Jezuit (U.S. Pat. No. 5,393,657) disclose a method to quantitate hamster DNA using the highly repetitive hamster Alu-equivalent consensus sequence as the target in a PCR assay. The readout for this assay relied on standard molecular biological techniques such as gel electrophoresis, hybridization techniques or sequencing. Anderson et al (US patent application 2009/0325175) also used hamster Alu-like sequences to detect residual DNA and relied on qPCR as the readout. A similar approach has been used to quantitate human DNA using human Alu elements as the target sequence for qPCR (Walker et al. Analytical Biochemistry 315 (2003) 122-128).

However, Alu-like sequences, and other apparently non-functional highly repetitive sequences, are not conserved across species and a separate assay using separate PCR primers is required for each species of DNA that must be tested.

Consequently, there is still a great need for a method having the advantages that qPCR brings to eukaryotic DNA quantitation such as low cost and high reproducibility, and the universality of the non-qPCR based Threshold Method.

### SUMMARY OF THE INVENTION

The inventors herein demonstrate that the 18S ribosomal RNA gene can be used as target sequence for quantitative PCR to detect and quantify residual genomic DNA from eukaryotic host cells used to produce recombinant proteins, in the low pictogram to high femtogram range.

Accordingly, in a first aspect, the present invention concerns a method of quantifying residual genomic DNA from a eukaryotic host cell in a recombinant protein product obtained from said host cell, wherein the method comprises:

a) purifying genomic DNA from a sample of the recombinant protein product, and optionally concentrating purified genomic DNA;

b) quantifying genomic DNA purified from the sample by quantitative real-time PCR amplification method using a pair of oligonucleotide primers targeting the 18S ribosomal RNA gene; and

c) determining the quantity of residual genomic DNA of eukaryotic host cell in the recombinant protein product.

Amplified sequences synthesized during quantitative real-time PCR in step b) may be detected using a non-specific DNA intercalating dye suitable for quantitative real-time PCR. In an embodiment, the DNA intercalating dye is selected from the group consisting of SYBR® Green I, SYBR® Green II, SYBR® Gold, BEBO, YO-PRO-1, LCGreen®, SYTO-9, SYTO-13, SYTO-16, SYTO-60, SYTO-62, SYTO-64, SYTO-82, POPO-3, TOTO-3, BOBO-3, TO-PRO-3, YO-PRO-1, PicoGreen® and SYTOX Orange. Preferably, the DNA intercalating dye is SYBR® Green I.

Alternatively, amplified sequences synthesized during quantitative real-time PCR in step b) may be detected using sequence-specific fluorogenic probes. In an embodiment, the sequence-specific fluorogenic probes are TaqMan probes or molecular beacons. Preferably, the sequence-specific fluorogenic probes are TaqMan probes.

In an embodiment, the pair of oligonucleotide primers targeting the 18S ribosomal RNA gene is constituted by a forward primer comprising, or consisting of, the sequence of SEQ ID No.: 1 and a reverse primer comprising, or consisting of, the sequence of SEQ ID No.: 2.

In another embodiment, amplified sequences synthesized during quantitative real-time PCR in step b) are detected using TaqMan probes comprising, or consisting of, a sequence of SEQ ID No.: 4.

The method of the invention may further comprise determining the purification yield of the genomic DNA of the sample by adding to said sample, before purification, a known quantity of synthetic control DNA whose sequence is not found in any known organism, and quantifying said control DNA simultaneously with genomic DNA using an additional pair of oligonucleotide primers specific of said control DNA during quantitative real-time PCR. The quantity of residual genomic DNA in the unpurified sample may be further determined by dividing the quantity of residual genomic DNA in the purified sample by the purification yield.

In an embodiment, the synthetic control DNA comprises, or consists of, the sequence of SEQ ID No.: 3. The synthetic control DNA may be amplified using a forward primer comprising, or consisting of, the sequence of SEQ ID No.: 5 and a reverse primer comprising, or consisting of, the sequence of SEQ ID No.: 6.

Amplified sequences of the synthetic control DNA synthesized during quantitative real-time PCR in step b) may be detected using a sequence-specific fluorogenic probe, preferably a TaqMan probe. In an embodiment, the sequence-specific fluorogenic probe is a TaqMan probe comprising, or consisting of, a sequence of SEQ ID No.: 7.

The host cell may be selected from the group consisting of human, monkey, mouse, rabbit, hamster, yeast, plant and insect cells.

The recombinant protein product may be a pharmaceutical composition or may be intended for pharmaceutical use.

In another aspect, the present invention concerns a kit for quantifying residual genomic DNA from a eukaryotic host cell in a recombinant protein product obtained from said host cell, wherein the kit comprises (i) at least one pair of primers targeting the 18S ribosomal RNA gene; and (ii) at least one synthetic control DNA molecule whose sequence is not found in any known organism, and optionally, a leaflet providing guidelines to use such a kit.

The kit may further comprise reagents needed for performing qPCR reaction and/or performing DNA purification. It may also further comprise multiple dilutions of standard DNA.

In an embodiment, the kit further comprises a pair of primers targeting the 18S ribosomal RNA gene constituted by a forward primer comprising, or consisting of, the sequence of SEQ ID No.: 1 and a reverse primer comprising, or consisting of, the sequence of SEQ ID No.: 2.

In another embodiment, the kit further comprises a synthetic control DNA molecule comprising, or consisting of, the sequence of SEQ ID No.: 3. The kit may also further comprise a pair of primers targeting the synthetic control DNA constituted by a forward primer comprising, or consisting of, the sequence 5′-AAGCGTGATATTGCTCTTTCGTATAG-3′ (SEQ ID No.: 5) and a reverse primer comprising, or consisting of, the sequence 5′-ACATAGCGACAGATTACAACATTAGTATTG-3′ (SEQ ID No.: 6).

In a further aspect, the present invention also concerns a use of the kit according to the invention to quantify residual genomic DNA from a eukaryotic host cell in a recombinant protein product obtained from said host cell.

### BRIEF DESCRIPTION OF THE DRAWINGS

FIG. 1: Amplification with the SybrGreen readout using DNA from seven eukaryotic species.

FIG. 2: Amplification with the TaqMan probe readout using DNA from seven eukaryotic species.

### DETAILED DESCRIPTION OF THE INVENTION

18S ribosomal RNA (rRNA) is the structural RNA for the small component of eukaryotic cytoplasmic ribosomes. Sequence data from 18S ribosomal RNA genes is widely used in molecular analysis to reconstruct the evolutionary history of organisms due to its slow evolutionary rate and the presence of highly conserved flanking regions allowing for the use of universal primers to access to gene sequences. Furthermore, 18S rRNA gene is considered as housekeeping gene that is expressed in all cells at relatively constant levels and is largely used as internal standard for RNA expression assays.

The inventors have herein surprisingly found that primers targeting the 18S rRNA gene can be used in qPCR assays to quantify residual genomic DNA from eukaryotic host cells used to produce recombinant proteins, in the low pictogram to high femtogram range.

Indeed, although the copy number of the 18S genes is much lower than the copy number of Alu-like sequences (hundreds of copies vs. hundreds of thousands of copies, i.e. a 1000-fold difference), the inventors demonstrate that the copy number of the 18S genes is enough to give sub-picogram sensitivity, as required by authorities for residual genomic DNA detection assays in recombinant protein drugs.

The 18S rRNA gene is present at 100 to 200 copies per genome in higher eukaryotes, such as mammals, and 7 copies in yeast, and is highly conserved across eukaryotes, allowing the same assay to be used for detection of residual DNA in proteins produced, for example, in yeast, mouse, human, hamster, rat and monkey cell lines, as well as in tumors grown in rabbits. About one copy of the 18S rRNA gene is present for each 20 million bases of eukaryotic genomic DNA and this allows for a highly sensitive assay that can detect DNA to as low as 200 femtograms in mammals and much lower in yeast.

Consequently, the method of the invention has the advantages that qPCR provide such as low cost, high reproducibility and ease of use, while also being universal for eukaryotes due to the fact that the 18S genes are highly conserves across species.

Accordingly, the present invention concerns a method of quantifying residual genomic DNA from a eukaryotic host cell in a recombinant protein product obtained from said host cell, wherein the method comprises:

a) purifying genomic DNA from a sample of the recombinant protein product, and optionally concentrating purified genomic DNA;

b) quantifying genomic DNA purified from the sample by quantitative real-time PCR amplification method using a pair of oligonucleotide primers targeting the 18S ribosomal RNA gene;

c) determining the quantity of residual genomic DNA of eukaryotic host cell in the recombinant protein product.

As used herein, the term “recombinant protein product” refers to a composition comprising a recombinant protein produced by a host cell, preferably by a eukaryotic host cell. The recombinant protein may be any protein produced by a host cell, preferably by a eukaryotic host cell. The protein may be for example an enzyme, an antibody or a fragment thereof, a receptor, a cytokine, a hormone, a coagulation factor or a growth factor. Preferably, the protein is intended for therapeutic or diagnostic use. In a particular embodiment, the recombinant protein product is a pharmaceutical composition or is intended for pharmaceutical use.

The sample of the recombinant protein product tested for residual genomic DNA can be any sample comprising the recombinant protein, such as for example a sample of the purified fraction of the recombinant protein, a sample of an intermediate fraction of the protein purification process, a sample of a composition comprising the purified recombinant protein and used to prepare the final drug formulation, or the final drug formulation comprising the recombinant protein.

As used herein, the term “residual genomic DNA” refers to any genomic DNA molecule from the host cell used to produce the recombinant protein and comprised in the product sample.

The eukaryotic host cell may be any eukaryotic host cell used to produce recombinant protein. In an embodiment, the host cell is selected from the group consisting of human, mouse, rabbit, monkey, hamster, yeast, plant and insect cells. In a preferred embodiment, the host cell is selected from the group consisting of human, mouse, hamster, rabbit, yeast, plant and insect cells.

Examples of human cells include, but are not limited to, HEK293 cells (ATCC CRL-1573). Examples of mouse cells include, but are not limited to, the mouse myeloma NSO cell line (ECACC 85110503). Examples of hamster cells include, but are not limited to, CHO-K1 cells (ATCC CCL-61). Examples of yeast cells include, but are not limited to, Pichia cells (ATCC 580) and Saccharomyces cerevisiae (ATCC 20626). Examples of plant cells include, but are not limited to, Nicotiana tabacum (ATCC 40904). Examples of insect cells include, but are not limited to, Sf9 cells (ATCC CRL-1711). Examples of monkey cells include, but are not limited to, Vero cells (ATCC CCL-81). The host cell may also be a rabbit cell, for example from a tumor producing monoclonal antibody and placed sub-cutaneouly in rabbits.

Prior to qPCR analysis, the residual genomic DNA is purified from the sample of the recombinant protein product, which usually consists of a high concentration of proteins, buffers, and stabilizers. Most of these non-DNA components are PCR inhibitors and should be eliminated.

Various methods known by the skilled person can be used to purify genomic DNA from the sample. In particular, genomic DNA may be purified using extraction-precipitation protocols, silica-membrane-based nucleic acid purification, magnetic-particle-based purification systems or DNA purification protocols using anion-exchange resin. Preferably, genomic DNA is purified from the sample using silica-membrane-based nucleic acid purification. Preferably, the method is chosen in order to remove PCR inhibitors such as divalent cations and proteins. Various genomic DNA purification kits using silica-membrane technology and suitable for PCR applications are commercially available such as for example QIAamp DNA Mini Kit (Qiagen).

In an embodiment, a proteinase, preferably Proteinase K, is added to the sample before purification to digest proteins contained in said sample. In another embodiment, an RNase, preferably RNase A, is added to the sample before purification to digest RNA contained in said sample. In a particular embodiment, a proteinase and an RNAse are added to the sample before purification to digest proteins and RNA contained in said sample. Conditions used to obtain optimal digestions are well known by the skilled person.

Purified genomic DNA is resuspended or eluted, depending on the purification technology used, in water or in a low salt or low EDTA buffer that is suitable for PCR applications.

The volume of DNA that can be used to the qPCR analysis is a few microliters at most. Consequently, if necessary, purified genomic DNA may be concentrated before PCR amplification. Any known method may be used to concentrate DNA, such as by filtration using a Microcon YM100 filter from Millipore corporation (Billerica, Ma, USA), or by ethanol precipitation.

Residual genomic DNA purified from the sample, and optionally concentrated, is then quantified using quantitative real-time PCR amplification method. Quantitative real-time PCR (or qPCR) is a well-known technique which is used to amplify and simultaneously quantify a targeted DNA molecule.

In qPCR, DNA amplification is monitored at each cycle of PCR by measuring a fluorescent signal. The amount of targeted sequence introduced into a qPCR reaction will determine the intensity of the signal generated during cycling, and by comparing this signal to that obtained from known standards, DNA can be accurately quantitated over a wide range of concentrations. Relative concentrations of DNA present during the exponential phase of the reaction are determined by plotting fluorescence against cycle number on a logarithmic scale. A threshold for detection of fluorescence above background is determined. The cycle at which the fluorescence from a sample crosses the threshold is called the cycle threshold, Cₜ. By using multiple dilutions of a known amount of standard DNA, a standard curve can be generated of log concentration against Cₜ. The amount of DNA in an unknown sample can then be calculated from its Cₜ value.

In the method of the invention, genomic DNA purified from the sample is quantified by qPCR using a pair of oligonucleotide primers targeting the 18S ribosomal RNA gene.

As used herein, the term “primer” refers to an oligonucleotide comprising from about 8 to about 35 bases in length, preferably from about 15 to about 30 bases in length, more preferably from about 15 to about 25 bases in length, and even more preferably of about 20 bases in length. As used in this specification, the term “about” refers to a range of values ±10% of the specified value. For example, “about 20” includes ±10% of 20, or from 18 to 22. Preferably, the term “about” refers to a range of values ±5% of the specified value.

The oligonucleotides suitable for use as primers targeting the 18S ribosomal RNA gene are directed to sequences of this gene that are repeated thought eukaryotic genomic DNA and are highly conserved across eukaryote species. Due to the extensive use of the 18S rRNA gene in phylogenetic analysis, various primers fulfilling these requirements are well known by the skilled person. For example, Torczynski et al (Nucleic Acid Research, vol 11, number 14, p 4879, 1983) compared the sequences of the rat, frog, and yeast 18S genes and found a high degree of similarity. Many regions of the gene are completely conserved and can be used to design universal primers. A number of primer pairs designed to conserved regions are now commercially available. For example Eurogentec (Liege, Belgium) sells a product for quantitating RNA by amplifying cDNA with primers to the 18S gene (catalogue number RT-CKFT-18S). A comparison of the sequences of these commercially available primers shows that they recognize regions of the 18S gene that are 100% conserved between frog, yeast, and rat. SABiosciences Corporation (Frederick, Md., USA) also sells a product with universal 18S primers (Product #PA-112). Furthermore, primer sequences to the 18S gene are also available in public databases such as the CalTech QPCR Table at http://www.its.caltech.edu/˜mirsky/qpcrprimer.htm.

In a particular embodiment the pair of oligonucleotide primers targeting the 18S ribosomal RNA gene is constituted by a forward primer comprising the sequence 5′-CGGCTACCACATCCAAGGAA-3′ (SEQ ID No.: 1) and a reverse primer comprising the sequence of 5′-GCTGGAATTACCGCGGCT-3′ (SEQ ID No.: 2). In a more particular embodiment, the forward primer consists of the sequence 5′-CGGCTACCACATCCAAGGAA-3′ (SEQ ID No.: 1) and the reverse primer consists of the sequence of 5′-GCTGGAATTACCGCGGCT-3′ (SEQ ID No.: 2). These primers are commercially available as mentioned above (Eurogentec, catalogue number RT-CKFT-18S). These primers are used to amplify a 155 bp region of the 18S rRNA gene.

In quantitative real-time PCR, DNA amplification is monitored at each cycle of PCR by measuring a fluorescent signal. Commonly, two methods may be used to detect amplified sequences: non-specific fluorescent dyes that intercalate with any double-stranded DNA, and sequence-specific DNA probes consisting of oligonucleotides that are labelled with a fluorescent reporter which permits detection only after hybridization of the probe with its complementary DNA target.

In an embodiment, amplified sequences synthesized during quantitative real-time PCR in step b) of the method of the invention are detected using a non-specific DNA intercalating dye. Numerous intercalating dyes have been developed to be suitable for quantitative real-time PCR (e.g. see Gudnason et al., Nucleic Acids Res. 2007 October; 35(19): e127) and any of these well-known dyes can be used in the present invention. In a particular embodiment, the DNA intercalating dye is selected from the group consisting of SYBR® Green I, SYBR® Green II, SYBR® Gold, BEBO, YO-PRO-1, LCGreen®, SYTO-9, SYTO-13, SYTO-16, SYTO-60, SYTO-62, SYTO-64, SYTO-82, POPO-3, TOTO-3, BOBO-3, TO-PRO-3, YO-PRO-1, PicoGreen® and SYTOX Orange. Preferably, the DNA intercalating dye is SYBR® Green I.

In another embodiment, amplified sequences synthesized during quantitative real-time PCR in step b) of the method of the invention are detected using sequence-specific fluorogenic probes. In this embodiment, amplified sequences are detected using oligonucleotide probes labeled with both a reporter fluorescent dye at one end and a quencher of fluorescence at the opposite end of the probe. The close proximity of the reporter to the quencher prevents detection of its fluorescence. Breaking the reporter-quencher proximity allows emission of fluorescence which can be detected after excitation with a laser.

Sequence-specific Probes based on different chemistries are available for real time detection. Preferably, sequence-specific fluorogenic probes are TaqMan® probes or molecular beacons.

Molecular beacons are single stranded hairpin shaped oligonucleotide probes. In the absence of a complementary target sequence, the beacon remains closed and there is no appreciable fluorescence. In the presence of a complementary target sequence, the “stem” portion of the beacon separates out resulting in the probe hybridizing to the target. When the beacon unfolds, the fluorophore is no longer quenched, and the molecular beacon fluoresces.

TaqMan® probes (Roche Molecular Systems, Inc.) are dual labelled hydrolysis probes consisting of a 18-22 bp oligonucleotide probe which is labelled with a reporter fluorophore at the 5′ end and a quencher fluorophore at the 3′ end. TaqMan® probes utilize the 5′ exonuclease activity of the enzyme Taq Polymerase for measuring the amount of target sequences in the PCR reaction. Till the time the probe is not hydrolysed, the quencher and the fluorophore remain in proximity to each other, separated only by the length of the probe. During PCR, the probe anneals specifically between the forward and reverse primer to an internal region of the amplified sequence. The polymerase then carries out the extension of the primer and replicates the template to which the TaqMan® probe is bound. The 5′ exonuclease activity of the polymerase cleaves the probe, releasing the reporter molecule away from the close vicinity of the quencher and resulting in an increase of the fluorescence intensity. Various fluorophores and quenchers are available to design TaqMan probes. Examples of fluorophores include, but are not limited to, NED (Life Technologies), 6-carboxyfluorescein (6-FAM), 5-carboxyfluorescein (5-FAM), tetrachlorofluorescin (TET), hexachloro-6-carboxyfluorescein (HEX), 6-Carboxy-4′,5′-dichloro-2′,7′-dimethoxyfluorescein, succinimidyl ester (JOE), [5-Carboxytetramethylrhodamine (TAMRASE) and carboxy-X-rhodamine (ROX). Examples of quenchers include, but are not limited to, tetramethylrhodamine (TAMRA), 4-(dimethylaminoazo)benzene-4-carboxylic acid (DABCYL), quenchers of the BHQ (Black Hole Quencher®) family, NFQ-MGB (non-fluorescent quencher and minor groove binder), QSY 21 carboxylic acid, succinimidyl ester, and QSY® 7 carboxylic acid, succinimidyl ester

Sequence-specific fluorogenic probes, in particular TaqMan probes, can be used in multiplex assays (for detection of several targeted sequences in the same qPCR reaction) using different fluorophores.

In a preferred embodiment, the sequence-specific fluorogenic probes are TaqMan probes.

In a particular embodiment, amplified sequences synthesized during quantitative real-time PCR in step b) of the method of the invention are detected using a TaqMan probe comprising, or consisting of, the sequence 5′-TTGGAGGGCAAGTCT-3′ (SEQ ID No.: 4). In a more particular embodiment, the reporter fluorophore at the 5′ end of this probe is NED dye and the quencher at the 3′ end is NFQ-MGB. Preferably, the TaqMan probe comprising the sequence 5′-TTGGAGGGCAAGTCT-3′ (SEQ ID No.: 4) is used to detect sequences amplifying by using the pair of oligonucleotide primers targeting the 18S ribosomal RNA gene constituted by a forward primer comprising, or consisting of, the sequence 5′-CGGCTACCACATCCAAGGAA-3′ (SEQ ID No.: 1) and a reverse primer comprising, or consisting of, the sequence of 5′-GCTGGAATTACCGCGGCT-3′ (SEQ ID No.: 2).

The skilled person knows how to conduct a quantitative real-time PCR reaction and how to analyze obtained data, i.e. how to quantify target DNA based on results of qPCR.

The person skilled in the art can easily adjust the parameters of the qPCR reaction such as the temperature and the length of each qPCR cycle, depending on various factors including the length of the target sequence to amplify, or the sequence and the length of the primers. A typical qPCR reaction comprises from about 20 cycles to about 50 cycles, preferably from about 25 cycles to about 45 cycles, and more preferably from about 30 cycles to about 45 cycles of PCR. Typically, each cycle comprises a denaturing step, for example 15 seconds at 95° C., followed by an annealing step, for example 1 minute at 60° C. The qPCR reaction can also comprise two additional initial steps, i.e. 2 minutes at 50° C. to allow degradation of carryover PCR products with Uracil-DNA Glycosylase and 1 minute at 95° C. to allow full denaturation of the template prior to amplification. Usually, qPCR reactions are performed using an automated system, e.g. Applied Biosystems 7500 instrument. Data analysis can also be done using a computer software, e.g. ABI Prism 7500 software.

Based on the quantification of genomic DNA purified from the sample by quantitative real-time PCR (step b) of the method of the invention), the quantity of residual genomic DNA from the eukaryotic host cell in the recombinant protein product can be easily determined (step c) by comparing the signal from the test sample with a standard curve made using genomic DNA from the same species. The DNA for the standard curve may be in a concentrated solution and may be quantitated by non-qPCR methods such as UV absorption, or picogreen staining.

During the genomic DNA purification process, there may be some sample to sample variation reducing the accuracy of assay as the final result obtained for a sample depends on both the amount of genomic DNA present in the initial sample and the purification yield. One approach to control for purification yield on a sample by sample basis is to spike, prior to purification, each sample with known amount of an inert control DNA sequence and to determine the amount of this control sequence in the purified product. The quantity of the control sequence and the quantity of residual genomic DNA from the host cell are then determined simultaneously in the same reaction by using duplexed qPCR.

Accordingly, in an embodiment, the method of the invention further comprises determining the purification yield of the genomic DNA of the sample by adding to said sample, before purification, a known quantity of synthetic control DNA whose sequence is not found in any known organism, and quantifying said control DNA simultaneously with genomic DNA using an additional pair of oligonucleotide primers specific of said control DNA during quantitative real-time PCR. The quantity of residual genomic DNA in the unpurified sample may be then determined by dividing the quantity of residual genomic DNA in the purified sample by the purification yield.

DNA sequences having no occurrence in any known organism will not cross react with DNA from the test sample and can be verified to be unique and not found in nature by comparing a potentially useful sequence with well known database. For example, any sequence can be compared with all known sequences by performing a Basic Local Alignment Search (BLAST) at http://blast.ncbi.nlm.nih.gov/Blast.cgi. Such sequences are described for example in Swango et al, Forensic Science International 158 (2006) 14-26). The person skilled in the art can easily choose such sequence and design specific primers to amplify it.

In a particular embodiment, the synthetic control DNA comprises, or consists of, the sequence 5′-AAGCGTGATATTGCTCTTTCGTATAGTTACCATGGCAATGC TTAGAACAATACTAATGTTGTAATCTGTCGCTATGT-3′ (SEQ ID No.: 3).

In this embodiment, the synthetic control DNA may be amplified using a forward primer comprising, or consisting of, the sequence 5′-AAGCGTGATATTGCTCTTTCGTATAG-3′ (SEQ ID No.: 5) and a reverse primer comprising, or consisting of, the sequence 5′-ACATAGCGACAGATTACAACATTAGTATTG-3′ (SEQ ID No.: 6).

In order to distinguish fluorescent signals providing from genomic DNA amplification and from synthetic control DNA amplification, amplified sequences are preferably detected using sequence-specific fluorogenic probes, in particular TaqMan probes, with distinct fluorophores. In a particular embodiment, the sequence-specific fluorogenic probe used to detect amplified sequences of the synthetic control DNA synthesized during qPCR reaction are detected using a TaqMan probe comprising, or consisting of, the sequence 5′-TACCATGGCAATGCT-3′ (SEQ ID No.: 7). In a more particular embodiment, the reporter fluorophore at the 5′ end of this probe is FAM and the quencher at the 3′ end is MGB.

In an alternative embodiment, the control DNA used to determine the purification yield of the genomic DNA is a prokaryotic DNA whose sequence is not found in any eukaryotic organism.

In a second aspect, the present invention concerns a method of quantifying residual genomic DNA from a prokaryotic host cell in a recombinant protein product obtained from said host cell, wherein the method comprises:

a) purifying genomic DNA from a sample of the recombinant protein product, and optionally concentrating purified genomic DNA;

b) quantifying genomic DNA purified from the sample by quantitative real-time PCR amplification method using a pair of oligonucleotide primers targeting the 16S ribosomal RNA gene;

c) determining the quantity of residual genomic DNA of prokaryotic host cell in the recombinant protein product.

The ribosome genes in prokaryotes differ in sequence from those in eukaryotes and the same primers specific to 18S rRNA as described above cannot be used. However, prokaryotes have multiple copies of the 16S ribosome gene which is highly conserved in sequence across strains. For example, E coli genomic DNA comprises 7 copies of this gene.

All the embodiments disclosed for the method of quantifying residual genomic DNA from a eukaryotic host cell are also contemplated in this method.

In a particular embodiment, the pair of oligonucleotide primers targeting the 16S ribosomal RNA gene is constituted by a forward primer comprising the sequence 5′-TCCTACGGGAGGCAGCAGT-3′ (SEQ ID No.: 8) and a reverse primer comprising the sequence of 5′-GGACTACCAGGGTATCTAATCCTGTT-3′ (SEQ ID No.: 9).

In a more particular embodiment, the primers listed above (SEQ ID No.: 8 and 9) are used to amplify an amplicon of the 16S rRNA gene of a prokaryotic host cell, preferably E. coli, in the presence of a non-specific DNA intercalating dye, such as SybrGreen, or sequence-specific fluorogenic probes, such as TaqMan probes, to give a sensitive and accurate assay for detecting residual prokaryotic genomic DNA, and in particular residual E. coli genomic DNA.

Residual DNA from prokaryotes may also contain plasmid DNA which is not detected with primers directed to sequences bacterial chromosome. To detect plasmid DNA, additional primers may be designed to sequence which is often conserved in plasmids such as the origin of replication of the parent plasmid PBR322 (most plasmids used for expression in E coli have this origin of replication). The assay for plasmid DNA can be done in a separate qPCR reaction using unlabelled primers with a non-specific DNA intercalating dye readout, preferably a SybrGreen readout, or can be done in a duplex qPCR reaction together with primers for genomic DNA using sequence-specific probes, preferably TaqMan probes.

In some circumstances a recombinant protein can be contaminated with genomic DNA from both prokaryotic and eukaryotic host cells. The assays described above can be combined either as two separate reactions, for example with a SybrGreen readout, or as a duplex reaction with sequence-specific fluorogenic probes such as TaqMan probes to detect both sources of contaminating DNA. The ability to detect both types of DNA is also useful as a means to quantitate the yield when DNA is purified from a sample prior to performing qPCR. For example, a sample containing eukaryotic DNA can be spiked with a known quantity of prokaryotic DNA and the purified sample is then tested for both prokaryotic and eukaryotic DNA. The amount of prokaryotic DNA recovered determines the yield, which can then be used to adjust the reading obtained for the eukaryotic DNA to have an accurate measure of the eukaryotic DNA in the original sample.

Accordingly, in another aspect, the invention concerns a method of quantifying residual genomic DNA from a eukaryotic and/or prokaryotic host cell(s) in a recombinant protein product obtained from said host cell(s), wherein the method comprises:

a) purifying genomic DNA from a sample of the recombinant protein product, and optionally concentrating purified genomic DNA;

b) quantifying genomic DNA purified from the sample by quantitative real-time PCR amplification method using a pair of oligonucleotide primers targeting the 18S ribosomal RNA gene and a pair of oligonucleotide primers targeting the 16S ribosomal RNA gene, amplified sequences being detected using sequence-specific fluorogenic probes;

c) determining the quantity of residual genomic DNA of eukaryotic host cell and/or prokaryotic host cell in the recombinant protein product.

All the embodiments disclosed for the methods of quantifying residual genomic DNA from a eukaryotic host cell and from a prokaryotic host cell are also contemplated in this method.

In another aspect, the present invention concerns a kit for quantifying residual genomic DNA from a eukaryotic host cell in a recombinant protein product obtained from said host cell, wherein the kit comprises (i) at least one pair of primers targeting the 18S ribosomal RNA gene; and (ii) at least one synthetic control DNA molecule whose sequence is not found in any known organism, and optionally, a leaflet providing guidelines to use such a kit.

The kit may also comprise reagents needed for performing qPCR reaction, such as DNA polymerase, and/or performing DNA purification.

The kit may further comprise multiple dilutions of standard DNA to generate a standard curve of log concentration against Cₜ.

In an embodiment, the kit comprises a pair of primers targeting the 18S ribosomal RNA gene constituted by a forward primer comprising, or consisting of, the sequence 5′-CGGCTACCACATCCAAGGAA-3′ (SEQ ID No.: 1) and a reverse primer comprising, or consisting of, the sequence 5′-GCTGGAATTACCGCGGCT-3′ (SEQ ID No.: 2).

In an embodiment, the kit comprises a synthetic control DNA molecule comprising, or consisting of, the sequence 5′-AAGCGTGATATTGCTCTTTCGTATAGTTACCATGGCAATGCTTAGAACAATAC TAATGTTGTAATCTGTCGCTATGT-3′ (SEQ ID No.: 3). The kit may further comprise a pair of primers targeting the synthetic control DNA constituted by a forward primer comprising, or consisting of, the sequence 5′-AAGCGTGATATTGCTCTTTCGTATAG-3′ (SEQ ID No.: 5) and a reverse primer comprising, or consisting of, the sequence 5′-ACATAGCGACAGATTACAACATAGTATTG-3′ (SEQ ID No.: 6).

The kit may further comprise sequence-specific fluorogenic probes, in particular TaqMan probes, designed to detect amplified sequences of genomic DNA and amplified sequences of control DNA.

The present invention further concerns a use of the kit as described above to quantify residual genomic DNA from a eukaryotic host cell in a recombinant protein product obtained from said host cell.

In a further aspect, the present invention concerns a kit for quantifying residual genomic DNA from a prokaryotic host cell in a recombinant protein product obtained from said host cell, wherein the kit comprises (i) at least one pair of primers targeting the 16S ribosomal RNA gene; and (ii) at least one synthetic control DNA molecule whose sequence is not found in any known organism, and optionally, a leaflet providing guidelines to use such a kit.

The kit may also comprise reagents needed for performing qPCR reaction, such as DNA polymerase, and/or performing DNA purification.

The kit may further comprise multiple dilutions of standard DNA to generate a standard curve of log concentration against Cₜ.

In an embodiment, the kit comprises a pair of primers targeting the 16S ribosomal RNA gene constituted by a forward primer comprising, or consisting of, the sequence 5′-TCCTACGGGAGGCAGCAGT-3′ (SEQ ID No.: 8) and a reverse primer comprising, or consisting of, the sequence of 5′-GGACTACCAGGGTATCTAATCCTGTT-3′ (SEQ ID No.: 9).

In an embodiment, the kit comprises a synthetic control DNA molecule comprising, or consisting of, the sequence 5′-AAGCGTGATATTGCTCTTTCGTATAGTTACCATGGCAATGCTAGAACAATAC TAATGTTGTAATCTGTCGCTATGT-3′ (SEQ ID No.: 3). The kit may further comprise a pair of primers targeting the synthetic control DNA constituted by a forward primer comprising, or consisting of, the sequence 5′-AAGCGTGATATTGCTCTTTCGTATAG-3′ (SEQ ID No.: 5) and a reverse primer comprising, or consisting of, the sequence 5′-ACATAGCGACAGATTACAACATTAGTATTG-3′ (SEQ ID No.: 6).

The kit may further comprise sequence-specific fluorogenic probes, in particular TaqMan probes, designed to detect amplified sequences of genomic DNA and amplified sequences of control DNA.

The present invention further concerns a use of the kit as described above to quantify residual genomic DNA from a prokaryotic host cell in a recombinant protein product obtained from said host cell.

The following examples are given for purposes of illustration and not by way of limitation.

### EXAMPLES

### Example 1

### Residual DNA Assays

Genomic DNA from various eukaryotic species was quantitated by qPCR using primers targeting the 18S rRNA gene.

The following primers were used to amplify a 155 bp region of the 18S ribosome gene: forward primer: CGGCTACCACATCCAAGGAA (SEQ ID No.: 1) and reverse primer GCTGGAATTACCGCGGCT (SEQ ID No.: 2).

The forward and reverse primers were used at a concentration of 100 nM.

qPCR reactions using SybrGreen readout were performed using SybrGreen Master mix from Applied Biosystems (Foster City, Calif., USA) according to supplier's instructions.

qPCR reactions were performed in an Applied Biosystems 7500 instrument with 25 μl reaction volumes. The following cycling parameters were used: 50° C. for 2 minutes, 95° C. for 10 minutes, and 40 cycles of denaturing at 95° C. for 15 seconds and annealing at 60° C. for 1 minute.

Each experiment was done in triplicate.

Human DNA Quantitation

Ten-fold dilutions of human DNA (Human DNA from Promega Corporation, Madison Wis., USA, catalogue #1471) from 20 ng to 200 fg were provided as template for qPCR reaction. The dilutions were done in distilled water just prior to addition to the mix reactions.

In a first experiment, 18S rRNA primers were used alone in a singleplex reaction with SybrGreen as the fluor. The Ct values obtained for this experiment are shown in Table 1.

TABLE 1
Human DNA detected with SybrGreen.

| DNA Amount (pg) | Ct Value |
|---|---|
| 20,000 | 17.5 |
| 2,000 | 21.0 |
| 200 | 24.5 |
| 20 | 27.3 |
| 2 | 31.2 |
| 0.2 | 34.6 |
| 0 | Not detected |

The slope of the standard curve is −3.55, which is in the expected range for qPCR.

In a second experiment, 18S rRNA primers were used with a TaqMan probe that binds within the amplified sequence to generate the fluorescent signal. The TaqMan probe was used at a concentration of 50 nM and had the following sequence: NED dye-TTGGAGGGCAAGTCT-MGB-NFQ (SEQ ID No.: 4) (Applied Biosystems).

The Ct values obtained during this assay are shown in Table 2.

TABLE 2
Human DNA detected with TaqMan Probe.

| DNA Amount (pg) | Ct Value |
|---|---|
| 20,000 | 21.0 |
| 2,000 | 24.3 |
| 200 | 27.6 |
| 20 | 31.2 |
| 2 | 34.5 |
| 0.2 | 38.0 |
| 0 | Not detected |

Quantitation of Genomic DNA from Various Eukaryotic Species

The 18S rRNA gene is conserved across species and the inventors demonstrated that both the SybrGreen and the TaqMan assays amplify genomic DNA from a number of eukaryotic sources.

Genomic DNA was purified from different eukaryotic sources by first digesting the proteins in the sample with Proteinase K and then by standard guanidinium/silica purification (Prep Seq Residual DNA Preparation Kit from Applied Biosystems). Genomic DNA was eluted from the silica using a minimal volume of a low salt/low EDTA buffer (50 μl).

Genomic DNA was purified from insect cells (Sf9 cells, ATCC CRL-1711), yeast cells (Pichia cells, ATCC 580) and human cells (HEK293 cells, ATCC CRL-1573)

Other genomic DNA were purchased from commercial suppliers as PCR ready genomic DNA (mouse, rabbit, hamster, plant (Arabidopsis thaliana) genomic DNA).

Conditions of qPCR reactions were the same as for human DNA assays as described above.

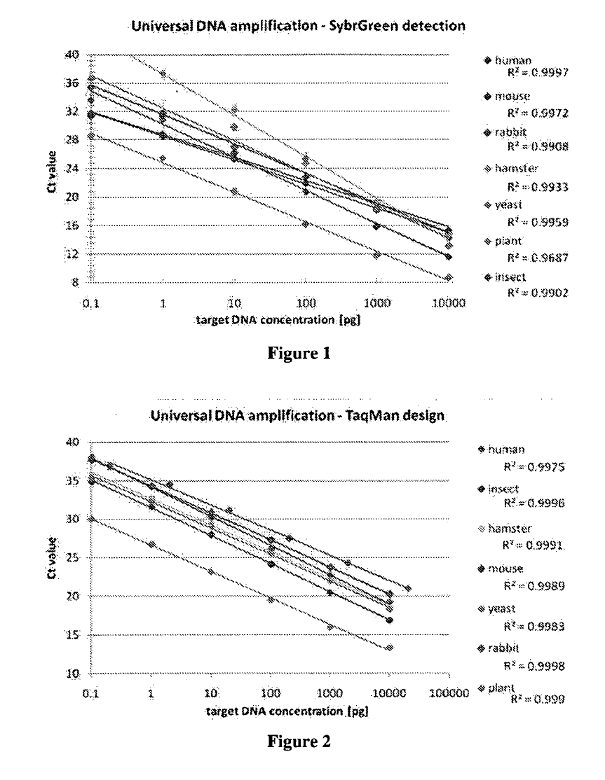

Results of qPCR reactions with the SybrGreen readout using DNA from seven eukaryotic species are shown in FIG. 1. The sensitivity for each assay extended to the low picogram level. Similar results were obtained with the TaqMan based assay, as shown in FIG. 2.

### Example 2

### Control DNA Spike

A control DNA is used to spike unpurified samples in order to determine purification yield. The control DNA is a synthetic oligonucleotide whose sequence is not found in any known organism and having the following sequence: AAGCGTGATATTGCTCTTTCGTATAGTTACCATGGCAATGCTAGAACAATAC TAATGTTGTAATCTGTCGCTATGT (SEQ ID No.: 3) (Swango et al, Forensic Science International 158 (2006) 14-26).

This DNA sequence is quantitated by qPCR using the following primers: forward primer: AAG CGT GAT ATT GCT CTT TCG TAT AG (SEQ ID No.: 5) and reverse primer ACA TAG CGA CAG ATT ACA ACA TTA GTA TTG (SEQ ID No.: 6), and the TaqMan probe having the following sequence: FAM-TAC CAT GGC AAT GCT-MGB-quencher (SEQ ID No.: 7).

qPCR reactions are done is the same conditions and parameters than in example 1.

This invention has been described with reference to various specific and exemplary embodiments and techniques. However, it should be understood that many variations and modifications will be obvious to those skilled in the art from the foregoing detailed description of the invention and be made while remaining within the spirit and scope of the invention.

1-24. (canceled)

25. A method of quantifying residual genomic DNA from a eukaryotic host cell in a recombinant protein product obtained from said host cell, wherein the method comprises:
a) purifying genomic DNA from a sample of the recombinant protein product, and optionally concentrating purified genomic DNA;
b) quantifying genomic DNA purified from the sample by quantitative real-time PCR amplification method using a pair of oligonucleotide primers targeting the 18S ribosomal RNA gene; and
c) determining the quantity of residual genomic DNA of eukaryotic host cell in the recombinant protein product.

26. The method according to claim 25, wherein amplified sequences synthesized during quantitative real-time PCR in step b) are detected using a non-specific DNA intercalating dye suitable for quantitative real-time PCR.

27. The method according to claim 26, wherein the DNA intercalating dye is selected from the group consisting of SYBR® Green I, SYBR® Green II, SYBR® Gold, BEBO, YO-PRO-1, LCGreen®, SYTO-9, SYTO-13, SYTO-16, SYTO-60, SYTO-62, SYTO-64, SYTO-82, POPO-3, TOTO-3, BOBO-3, TO-PRO-3, YO-PRO-1, PicoGreen® and SYTOX Orange.

28. The method according to claim 26, wherein the DNA intercalating dye is SYBR® Green I.

29. The method according to claim 25, wherein amplified sequences synthesized during quantitative real-time PCR in step b) are detected using sequence-specific fluorogenic probes.

30. The method according to claim 29, wherein the sequence-specific fluorogenic probes are TaqMan probes or molecular beacons.

31. The method according to claim 29, wherein the sequence-specific fluorogenic probes are TaqMan probes.

32. The method according to claim 25, wherein the pair of oligonucleotide primers targeting the 18S ribosomal RNA gene is constituted by a forward primer comprising or consisting of the sequence of SEQ ID NO: 1 and a reverse primer comprising or consisting of the sequence of SEQ ID NO: 2.

33. The method according to claim 25, wherein amplified sequences synthesized during quantitative real-time PCR in step b) are detected using TaqMan probes comprising or consisting of a sequence of SEQ ID NO: 4.

34. The method according to claim 25, further comprising determining the purification yield of the genomic DNA of the sample by adding to said sample, before purification, a known quantity of synthetic control DNA whose sequence is not found in any known organism, and quantifying said control DNA simultaneously with genomic DNA using an additional pair of oligonucleotide primers specific of said control DNA during quantitative real-time PCR.

35. The method according to claim 34, further comprising determining the quantity of residual genomic DNA in the unpurified sample by dividing the quantity of residual genomic DNA in the purified sample by the purification yield.

36. The method according to claim 34, wherein the synthetic control DNA comprises or consists of the sequence of SEQ ID NO: 3.

37. The method according to claim 36, wherein the synthetic control DNA is amplified using a forward primer comprising or consisting of the sequence of SEQ ID NO: 5 and a reverse primer comprising or consisting of the sequence of SEQ ID NO: 6.

38. The method according to claim 37, wherein amplified sequences of the synthetic control DNA synthesized during quantitative real-time PCR in step b) are detected using a sequence-specific fluorogenic probe.

39. The method according to claim 38, wherein the sequence-specific fluorogenic probe is a TaqMan probe comprising or consisting of a sequence of SEQ ID NO: 7.

40. The method according to claim 25, wherein the host cell is selected from the group consisting of human, mouse, monkey, rabbit, hamster, yeast, plant and insect cells.

41. The method according to claim 25, wherein the recombinant protein product is a pharmaceutical composition or is intended for pharmaceutical use.

42. A kit for quantifying residual genomic DNA from a eukaryotic host cell in a recombinant protein product obtained from said host cell, wherein the kit comprises (i) at least one pair of primers targeting the 18S ribosomal RNA gene; and (ii) at least one synthetic control DNA molecule whose sequence is not found in any known organism, and optionally, a leaflet providing guidelines to use such a kit.

43. The kit according to claim 42, further comprising reagents needed for performing qPCR reaction and/or performing DNA purification.

44. The kit according to claim 42, further comprising multiple dilutions of standard DNA and, optionally, reagents needed for performing qPCR reaction and/or performing DNA purification.

45. The kit according to claim 42, wherein the kit further comprises a pair of primers targeting the 18S ribosomal RNA gene constituted by a forward primer comprising, or consisting of, the sequence of SEQ ID NO: 1 and a reverse primer comprising, or consisting of, the sequence of SEQ ID NO: 2.

46. The kit according to claim 42, wherein the kit further comprises a synthetic control DNA molecule comprising, or consisting of, the sequence of SEQ ID NO: 3.

47. The kit according to claim 46, further comprising a pair of primers targeting the synthetic control DNA constituted by a forward primer comprising, or consisting of, the sequence 5′-AAGCGTGATATTGCTCTTTCGTATAG-3′ (SEQ ID NO: 5) and a reverse primer comprising, or consisting of, the sequence 5′-ACATAGCGACAGATTACAACATTAGTATTG-3′ (SEQ ID NO: 6).


SEQUENCE LISTING (9 sequences)

SEQ ID NO: 1 - DNA, 20 bp, Artificial
  18S forward primer
cggctaccacatccaaggaa

SEQ ID NO: 2 - DNA, 17 bp, Artificial
  18S reverse primer
gctggaattaccgcggc

SEQ ID NO: 3 - DNA, 77 bp, Artificial
  synthetic control DNA
aagcgtgatattgctctttcgtatagttaccatggcaatgcttagaacaatactaatgttgtaatctgtcgctatgt

SEQ ID NO: 4 - DNA, 15 bp, Artificial
  18S TaqMan probe
ttggagggcaagtct

SEQ ID NO: 5 - DNA, 26 bp, Artificial
  control DNA forward primer
aagcgtgatattgctctttcgtatag

SEQ ID NO: 6 - DNA, 30 bp, Artificial
  Control DNA reverse primer
acatagcgacagattacaacattagtattg

SEQ ID NO: 7 - DNA, 15 bp, Artificial
  Control DNA TaqMan probe
taccatggcaatgct

SEQ ID NO: 8 - DNA, 19 bp, Artificial
  16S forward primer
tcctacgggaggcagcagt

SEQ ID NO: 9 - DNA, 26 bp, Artificial
  16S reverse primer
ggactaccagggtatctaatcctgtt